(19) United States
(12) Patent Application Publication
(10) Pub. No.: US 2014/0234345 A1
(43) Pub. Date: Aug. 21, 2014

(54) NOVEL ANTI-CANCER ISOCARBOSTYRIL ALKALOID CONJUGATES

(71) Applicants: Claude Mercure, St. Catharines (CA); Rajashree Sathyamurthy, Westford, MA (US)

(72) Inventors: Claude Mercure, St. Catharines (CA); Rajashree Sathyamurthy, Westford, MA (US)

(73) Assignee: BIOLYSE PHARMA CORPORATION, St. Catharines, ON (CA)

(21) Appl. No.: 14/131,950

(22) PCT Filed: Jul. 11, 2012
(86) PCT No.: PCT/CA2012/050473

§ 371 Date: May 5, 2014

Related U.S. Application Data
(60) Provisional application No. 61/506,386, filed on Jul. 11, 2011.

(51) Int. Cl. A61K 47/48; A61K 31/4741

(52) U.S. Cl. CPC A61K 47/48384; A61K 31/4741; A61K 47/48038
USPC 424/181.1; 546/65; 514/287; 530/391.7

(57) ABSTRACT

The present application is directed to covalent conjugates between an isocarbostyril alkaloid and a lipophilic biomolecule, to pharmaceutical compositions comprising the conjugates and to therapeutic uses thereof, in particular for treating cancer.

### FIELD

The present application is in the field of anti-cancer therapeutics. In particular, the present application is directed to new lipophilic biomolecule conjugates of isocarbostyril alkaloids having surprisingly improved activity along with improved solubility and bioavailability, as well as pharmaceutical compositions comprising these conjugates and therapeutic uses thereof, in particular for treating cancer.

### BACKGROUND

Plants in the Amarylliadaceae species have been known for their anti-tumour effects since the era of the ancient Greeks. It was reported that the most likely candidates that cause these effects were the isocarbostyril components of these plants. It is now evident that narciclasine, pancrastatin and other isocarbostyrils of the Amaryllidaceae species have shown anti-cancer activity. It has been the goal of researchers to develop these compounds for use against a wide range of cancers, but there have been many issues concerning their development. As with most anti-cancer drugs these problems relate to the drugs' solubility, activity and selectivity towards cancer cells.

A large percentage of cancer patients die from metastases or from tumour invasion of vital organs. Unsuccessful treatment results are mostly due to resistance to natural cell death or apoptosis in numerous cancerous cell lines. This is especially evident when the disease has evolved to a metastatic form. Most of the agents used today by clinicians to combat cancer are ineffective in combating apoptosis-resistant metastatic cancers. Narciclasine has shown evidence that it has selective ability to kill apoptosis-resistant and apoptosis-sensitive cancer cells as well as having anti proliferative and anti-migratory effect

### SUMMARY

It has surprisingly been found that conjugation of a lipophilic biomolecule to an isocarbostyril alkaloid provides compounds that have improved anti-tumour activity compared to the unconjugated parent molecule.

Accordingly, the present application includes a covalent conjugate of one or more lipophilic biomolecules with an isocarbostyril alkaloid, wherein the one or more lipophilic biomolecules are covalently linked to the isocarbostyril alkaloid via a linker group, or a pharmaceutically acceptable salt and/or hydrate thereof.

In an embodiment, the isocarbostyril alkaloid is selected from narciclasine, pancratistatin and lycoricidine, or a derivative thereof.

In an embodiment, the present application includes a compound of Formula I:

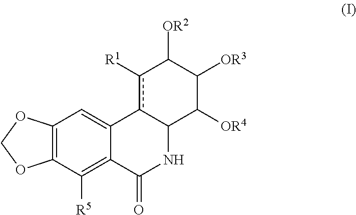

wherein
R¹ and R⁵, are independently, H, OH or O-(L)ₙ-B;
R², R³ and R⁴, are independently, H or -(L)ₙ-B;
provided that one, two or three of R¹, R², R³, R⁴ and R⁵ comprise -(L)ₙ-B;
L is a linker group;
B is a lipophilic biomolecule; and [symbol]
is a single or a double bond,
or a stereoisomer thereof, or a pharmaceutically acceptable salt and/or solvate.

In an embodiment, B is a long chain fatty acid. In a further embodiment, B is a monoclonal antibody.

The present application also includes a pharmaceutical composition comprising one or more compounds of the application and a pharmaceutically acceptable carrier.

In a further embodiment, the compounds of the present application are used as medicaments. Accordingly the application also includes a compound of the application for use as a medicament.

The compounds of the application have been shown to have anti-tumour activity against a wide variety of tumour cell lines. Therefore these compounds are useful as anti-tumour agents for treating cancer. Accordingly, the present application also includes a method for treating cancer comprising administering a therapeutically effective amount of one or more compounds of the application to a subject in need thereof. In an embodiment of the application, the cancer is selected from pancreatic cancer, lung cancer, colorectal cancer, prostate cancer, breast cancer and cervical cancer.

Other features and advantages of the present disclosure will become apparent from the following detailed description. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

### BRIEF DESCRIPTION OF THE DRAWINGS

The application will now be described in greater detail with reference to the drawings in which:

FIG. 1 is a graph showing the effect of narciclasine and compound I(a) on six different cancer cell lines after 48 hours.

FIG. 2 is a graph showing the effect of narciclasine and compound I(a) on six different cancer cell lines after 72 hours.

FIG. 3 is a graph showing the effect of compound I(a) in vitamin E on six different cancer cell lines after 48 hours.

FIG. 4 is a graph showing the effect of compound I(a) in vitamin E on six different cancer cell lines after 72 hours.

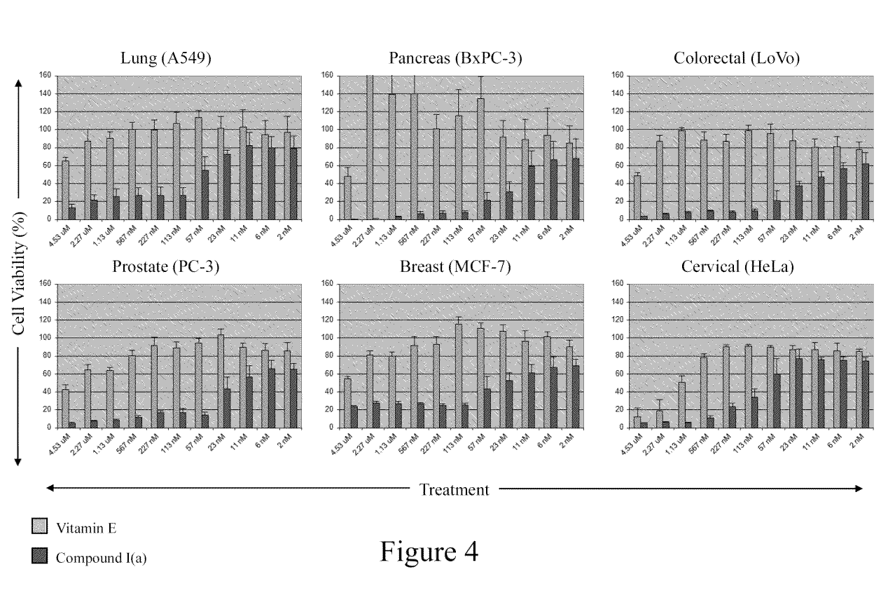

FIG. 5 is a graph showing the effect of compound I(a) and narciclasine in DMSO and compound I(a) in vitamin E against healthy PBMC cells.

### DESCRIPTION OF VARIOUS EMBODIMENTS

### I. Definitions

Unless otherwise indicated, the definitions and embodiments described in this and other sections are intended to be applicable to all embodiments and aspects of the application herein described for which they are suitable as would be understood by a person skilled in the art.

As used in this application, the singular forms “a”, “an” and “the” include plural references unless the content clearly dictates otherwise. For example, an embodiment including “a compound” should be understood to present certain aspects with one compound, or two or more additional compounds.

In embodiments comprising an “additional” or “second” component, such as an additional or second compound, the second component as used herein is chemically different from the other components or first component. A “third” component is different from the other, first, and second components, and further enumerated or “additional” components are similarly different.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

The term “cancer” as used herein means a metastatic and/or a non-metastatic cancer, and includes primary and secondary cancers. Reference to cancer includes reference to cancer cells.

The term “alkyl” as used herein, whether it is used alone or as part of another group, means straight or branched chain, saturated alkyl groups.

The term “alkylene” as used herein, whether alone or as part of another group, means an alkyl group that is bivalent, i.e. that it is substituted on two ends with another group.

The term “compounds of the application” or “compounds of the present application” as used herein refers to a compound of Formula I, an stereoisomer thereof, or a pharmaceutically acceptable salt and/or hydrate thereof.

The term “narciclasine” as used herein refers to the compound:

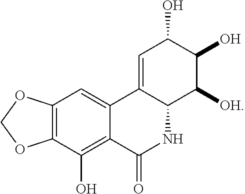 [SMILES: O=C1N[C@@H]2C(=C[C@H](O)[C@@H](O)[C@H]2O)c2cc3c(c(O)c21)OCO3]

The term “pancratistatin” as used herein refers to the compound:

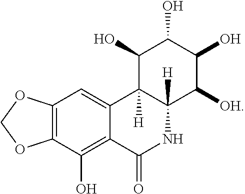

The term “lycoricidine” as used herein refers to the compound:

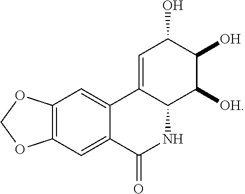 [SMILES: O=C1N[C@@H]2C(=C[C@H](O)[C@@H](O)[C@H]2O)c2cc3c(cc21)OCO3]

The term “derivative” as used herein refers to a compound that is derived from a parent compound by modification of one or more or the functional groups in the parent molecule. For example, a derivative of narciclasine may be a compound where one of more of the hydroxyl groups has been removed or converted to, for example, a C₁₋₄alkyl ether, C₁₋₄alkyl ester, an arylether, an arylester or a ketone, or the amide group has been reduced or derivatized with, for example, a C₁₋₄alkyl group. Other derivatives of isocarbostryil alkaloids, included within the scope of the present application, are described in WO 2008/043846, the contents of which are incorporated by reference.

The term “fatty acid” as used herein refers to a carboxylic acid with a long unbranched aliphatic tail (chain) which is either saturated or unsaturated. “Long” chain fatty acids have, 12 to 28, suitably, 12 to 22 carbon atoms in the chain.

The terms “protective group” or “protecting group” or “PG” or the like as used herein refer to a chemical moiety which protects or masks a reactive portion of a molecule to prevent side reactions in those reactive portions of the molecule, while manipulating or reacting a different portion of the molecule. After the manipulation or reaction is complete, the protecting group is removed under conditions that do not degrade or decompose the remaining portions of the molecule. The selection of a suitable protecting group can be made by a person skilled in the art. Many conventional protecting groups are known in the art, for example as described in “Protective Groups in Organic Chemistry” McOmie, J. F. W. Ed., Plenum Press, 1973, in Greene, T. W. and Wuts, P. G. M., “Protective Groups in Organic Synthesis”, John Wiley & Sons, 3^{rd} Edition, 1999 and in Kocienski, P. Protecting Groups, 3rd Edition, 2003, Georg Thieme Verlag (The Americas). Examples of suitable protecting groups include, but are not limited to t-Boc, Ac, Ts, Ms, silyl ethers such as TMS, TBDMS, TBDPS, Tf, Ns, Bn, Fmoc, benzoyl, dimethoxytrityl, methoxyethoxymethyl ether, methoxymethyl ether, pivaloyl, p-methyoxybenzyl ether, tetrahydropyranyl, trityl, ethoxyethyl ethers, carbobenzyloxy, benzoyl and the like.

The term “subject” as used herein includes all members of the animal kingdom including mammals, and suitably refers to humans.

The term “pharmaceutically acceptable salt” means an acid addition salt or a base addition salt which is suitable for, or compatible with, the treatment of patients.

The term “pharmaceutically acceptable acid addition salt” as used herein means any non-toxic organic or inorganic salt of any basic compound. Basic compounds that form an acid addition salt include, for example, compounds comprising an thiol group. Illustrative inorganic acids which form suitable salts include hydrochloric, hydrobromic, sulfuric and phosphoric acids, as well as metal salts such as sodium monohydrogen, orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono or di-acid salts can be formed, and such salts may exist in either a hydrated, solvated or substantially anhydrous form. In general, acid addition salts are more soluble in water and various hydrophilic organic solvents, and generally demonstrate higher melting points in comparison to their free base forms. The selection of the appropriate salt will be known to one skilled in the art.

The term “pharmaceutically acceptable base addition salt” as used herein means any non-toxic organic or inorganic base addition salt of any acidic compound. Acidic compounds that form a basic addition salt include, for example, compounds comprising a carboxylic acid group. Illustrative inorganic bases which form suitable salts include lithium, sodium, potassium, calcium, magnesium or barium hydroxide. Illustrative organic bases which form suitable salts include aliphatic, alicyclic or aromatic organic amines such as methylamine, trimethylamine and picoline, alkylammonias or ammonia. The selection of the appropriate salt will be known to a person skilled in the art.

The formation of a desired compound salt is achieved using standard techniques. For example, the neutral compound is treated with an acid or base in a suitable solvent and the formed salt is isolated by filtration, extraction or any other suitable method.

The term “solvate” as used herein means a compound or its pharmaceutically acceptable salt, wherein molecules of a suitable solvent are incorporated in the crystal lattice. A suitable solvent is physiologically tolerable at the dosage administered. Examples of suitable solvents are ethanol, water and the like. When water is the solvent, the molecule is referred to as a “hydrate”. The formation of solvates will vary depending on the compound and the solvate. In general, solvates are formed by dissolving the compound in the appropriate solvent and isolating the solvate by cooling or using an antisolvent. The solvate is typically dried or azeotroped under ambient conditions.

In embodiments of the application, the compounds described herein have at least one asymmetric centre. Where compounds possess more than one asymmetric centre, they may exist as diastereomers. It is to be understood that all such isomers and mixtures thereof in any proportion are encompassed within the scope of the present application. It is to be further understood that while the stereochemistry of the compounds may be as shown in any given compound listed herein, such compounds may also contain certain amounts (e.g. less than 50%, suitably less than 20%, more suitably less than 10%, more suitably less than 5%) of compounds of the application having alternate stereochemistry.

The term “treating” or “treatment” as used herein and as is well understood in the art, means an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, diminishment of the reoccurrence of disease, and remission (whether partial or total), whether detectable or undetectable. “Treating” and “Treatment” can also mean prolonging survival as compared to expected survival if not receiving treatment. “Treating” and “treatment” as used herein also include prophylactic treatment. For example, a subject with early stage cancer can be treated to prevent progression or metastases, or alternatively a subject in remission can be treated with a compound of the application to prevent recurrence. Treatment methods comprise administering to a subject a therapeutically effective amount of a compound of the application and optionally consists of a single administration, or alternatively comprises a series of applications. For example, the compounds of the application may be administered at least once a week. However, in another embodiment, the compounds may be administered to the subject from about one time per three weeks, or about one time per week to about once daily for a given treatment. In another embodiment, the compound is administered 2, 3, 4, 5 or 6 times daily. The length of the treatment period depends on a variety of factors, such as the severity of the disease, the age of the patient, the concentration, the activity of the compounds of the application, and/or a combination thereof. It will also be appreciated that the effective dosage of the compound used for the treatment or prophylaxis may increase or decrease over the course of a particular treatment or prophylaxis regime. Changes in dosage may result and become apparent by standard diagnostic assays known in the art. In some instances, chronic administration may be required. For example, the compounds are administered to the subject in an amount and for a duration sufficient to treat the patient.

As used herein, the term “effective amount” or “therapeutically effective amount” means an amount effective, at dosages and for periods of time necessary to achieve the desired result. For example in the context or treating a cancer, an effective amount is an amount that, for example, induces remission, reduces tumor burden, and/or prevents tumor spread or growth compared to the response obtained without administration of the compound. Effective amounts may vary according to factors such as the disease state, age, sex, weight of the subject. The amount of a given compound that will correspond to such an amount will vary depending upon various factors, such as the given drug or compound, the pharmaceutical formulation, the route of administration, the type of disease or disorder, the identity of the subject or host being treated, and the like, but can nevertheless be routinely determined by one skilled in the art.

The term “administered” as used herein means administration of a therapeutically effective dose of a compound or composition of the application to a cell either in cell culture or in a patient.

### II. Compounds of the Application

To improve its solubility, bioavailability and binding with pharmaceutical carriers, the naturally occurring isocarbostyril alkaloid, narciclasine was modified to contain a biomolecular lipophilic group. In one example, the biomolecular lipophilic group was added to narciclasine via conjugation with docosahexaenoic acid (DHA), however, any long chain fatty acid (C₁₂₋₂₈, suitably C₁₂₋₂₂) such as oleic acid and linolenic acid, would be a suitable lipophilic group as such biomolecules preferentially bind potential carriers, can cross the blood-brain barrier and cell membranes thus enhancing the bioavailability of the drug. Omega 3 fatty acids are also well known to have positive health effects such as lowering blood tryglycerides and reducing heart disease and have been reported to have possible benefits in fighting certain cancers.

Accordingly, the present application includes a covalent conjugate of one or more lipophilic biomolecules with an isocarbostyril alkaloid, wherein the one or more lipophilic biomolecules are covalently linked to the isocarbostyril alkaloid via a linker group, or a pharmaceutically acceptable salt and/or hydrate thereof.

In an embodiment, the isocarbostyril alkaloid is selected from narciclasine, pancratistatin and lycoricidine, or a derivative thereof.

In an embodiment, the present application includes a compound of Formula I:

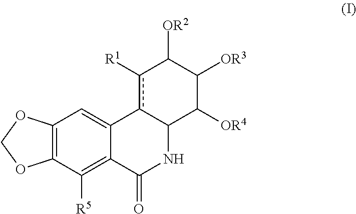

wherein
R¹ and R⁵, are independently, H, OH or O-(L)ₙ-B;
R², R³ and R⁴, are independently, H or -(L)ₙ-B;
provided that one, two or three of R¹, R², R³, R⁴ and R⁵ comprise -(L)ₙ-B;
L is a linker group;
B is a lipophilic biomolecule; and [symbol]
is a single or a double bond,
or a stereoisomer thereof, or a pharmaceutically acceptable salt and/or solvate.

In an embodiment of the application, when [symbol] is a double bond, R¹ is H.

In an embodiment of the application R¹ is H, R² is O-(L)ₙ-B, R³ is OH, R⁴ is OH, R⁵ is OH and [symbol] is a double bond to provide narciclasine 2-position conjugate. In another embodiment of the application R¹ is H, R² is OH, R³ is OH, R⁴ is OH, R⁵ is O-(L)ₙ-B and [symbol] is a double bond to provide a narciclasine 7-position conjugate.

In an embodiment of the application, R¹ is H, R² is O-(L)ₙ-B, R³ is OH, R⁴ is OH, R⁵ is and [symbol] is a double bond to provide lycoridine 2-position conjugate.

In an embodiment of the application, R¹ is OH, R² is O-(L)ₙ-B, R³ is OH, R⁴ is OH, R⁵ is OH and [symbol] is a single bond to provide pancratistatin 2-position conjugate. In another embodiment of the application R¹ is OH, R² is OH, R³ is OH, R⁴ is OH, R⁵ is O-(L)ₙ-B and [symbol] is a single bond to provide a pancratistatin 7-position conjugate.

In another embodiment of the application, the relative stereochemistry of the compounds of Formula I is that of the natural isocarbostril alkaloids. Accordingly, the compound of Formula I has the following relative stereochemistry when [symbol] is a double bond:

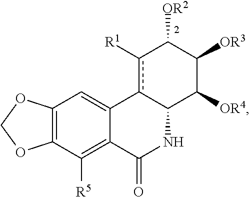

and the following relative stereochemistry when [symbol] is a single bond:

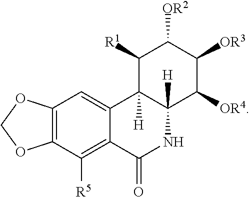

In the above structures, it is an embodiment, that the compounds comprise up to 50% of a compound having an alternate stereochemistry, in particular at the C2 position.

A person skilled in the art would appreciate that the lipophilic biomolecule (B) can optionally be linked to the isocarbostyril alkaloid using any suitable linker group. In an embodiment, the linker group (L) is selected from C₁₋₂₀alkylene in which one or more of the CH₂ groups is optionally replaced with O, NH, S, NC₁₋₄alkyl, C(O), C(O)O, C(O)NH, C(O)N(C₁₋₄)alkyl, S(O), SO₂, S(O)—NH, S(O)N(C₁₋₄alkyl), SO₂—NH and SO₂—N(C₁₋₄alkyl). In a further embodiment, L is selected from C₁₋₁₀alkylene in which one or more of the CH₂ groups is optionally replaced with C(O)O or O. In a further embodiment, L is selected from C₁₋₆alkylene in which one or more of the CH₂ groups is optionally replaced with C(O)O.

In an embodiment n is 0 (i.e., the optional linker is not present).

In an embodiment, n is 1 (i.e. the optional linker is present).

In an embodiment, B is a long chain fatty acid. In another embodiment, the fatty acid is a C₁₂₋₂₂ fatty acid. In a further embodiment, B is selected from cis-docosahexaenoic acid (DHA), oleic acid, lauric acid, palmitic acid, linoelaidic acid, vaccenic acid, palmitoleic, myristoleic, a-linolenic acid (ALA), arachidonic acid, eicosapentaenoic acid (EPA) and eruric acid. In a further embodiment, B is selected from cis-docosahexaenoic acid (DHA), EPA, ALA, lauric acid and oleic acid. In another embodiment, B is DHA.

In an embodiment of the application, one or two of R¹, R², R³, R⁴ and R⁵ comprises -(L)ₙ-B. In another embodiment one of R¹, R², R³, R⁴ and R⁵ comprises -(L)ₙ-B. In another embodiment R² is -(L)ₙ-B.

In another embodiment, B is a monoclonal antibody which would deliver the compound to cancer cells specifically. These monoclonal antibodies are directed towards cancer-related antigens such as, but not limited to, CD20, CD22, and the Interlukin-2 receptor.

The preparation of the compounds of the application can be performed using methods known in the art. For example, the fatty acid may be coupled with the isocarbostyril alkaloid using standard carboxylic acid activation, nucleophilic coupling. In an embodiment, one or more of the free hydroxyl groups on the alkaloid are protected with a suitable protecting group prior to coupling. The selection of suitable protecting groups would be well within the skill of a person in the art. When a linker group is present, the linker may be first attached to either the carboxylic acid or the alkaloid prior to forming the conjugate. In this case, one or more of the free hydroxyl groups may be reacted with a linker group precursor comprising a leaving group in a nucleophilic substitution reaction. The linker group precursor may comprise the biomolecular lipophilic group, or alternatively, the biomolecular linker group may be added to the alkaloid-linker molecule.

Both the isocarbostyril alkaloid and the fatty acids may be isolated from natural sources or may be chemically synthesized using methods known in the art. Methods of preparing isocarbostyril alkaloid derivatives is described, for example, in WO 2008/043846, the contents which are incorporated herein by reference.

Methods of preparing monoclonal antibodies are known in the art.

### III. Compositions of the Application

The compounds of the application are suitably formulated into pharmaceutical compositions for administration to subjects in a biologically compatible form suitable for administration in vivo. Accordingly, the present application also includes a pharmaceutical composition comprising one or more compounds of the application and a pharmaceutically acceptable carrier.

In an embodiment of the application, the compounds of the application are formulated into a composition comprising vitamin E, for example, vitamin E d-alpha-tocopheryl polyethylene glycol 1000 succinate (Vitamin E TPGS), as a carrier. Vitamin E TPGS is both fat and water soluble and is able to dissolve the compounds of the application to make a true molecular solution where the particle size of the compound of the application is below the human visible range. The vitamin E TPGS solution of the compounds of the application can be used, for example, in the formation of a pharmaceutical composition to be used as an injectable. Accordingly, the present application also includes a composition comprising one or more compounds of the application and Vitamin E TPGS. In an embodiment, the ratio (w/w) of the one or more compounds of the application to Vitamin E TPGS is about 1:50 to about 1:10, about 1:20 to about 1:15 or about 3:20.

In another embodiment of the application, the compounds of the application are formulated into a composition comprising Cremophor™ EL (polyethoxylated castor oil) and dehydrated ethanol as the carrier. Cremophor EL is a nonionic emulsifier that is used to assist in the solubilization of hydrophobic drugs. The polyethoxylated castor oil/ethanol solution of the compounds of the application can be used, for example, in the formation of a pharmaceutical composition to be used as an injectable. Accordingly, the present application also includes a composition comprising one or more compounds of the application, polyethoxylated castor oil and ethanol. In an embodiment, the ratio (w/w) of the one or more compounds of the application to polyethoxylated castor oil is about 1:100 to about 1:10, about 1:80 to about 1:30 or about 1:60. In a further embodiment, the ratio (v/v) of polyethoxylated castor oil to ethanol is about 1:2 to about 2:1, or about 1:1.

The compounds of the application, may be administered to a subject in a variety of forms depending on the selected route of administration, as will be understood by those skilled in the art. A compound of the application, may be administered, for example, by oral, parenteral, buccal, sublingual, nasal, rectal, patch, pump or transdermal administration and the pharmaceutical compositions formulated accordingly. Parenteral administration includes intravenous, intraperitoneal, subcutaneous, intramuscular, transepithelial, nasal, intrapulmonary, intrathecal, rectal and topical modes of administration. Parenteral administration may be by continuous infusion over a selected period of time. Conventional procedures and ingredients for the selection and preparation of suitable compositions are described, for example, in Remington's Pharmaceutical Sciences (2000-20th edition) and in The United States Pharmacopeia: The National Formulary (USP 24 NF19) published in 1999.

A compound of the application, may be orally administered, for example, with an inert diluent or with an assimilable edible carrier, or it may be enclosed in hard or soft shell gelatin capsules, or it may be compressed into tablets, or it may be incorporated directly with the food of the diet. For oral therapeutic administration, the compound may be incorporated with excipient and used in the form of ingestible tablets, buccal tablets, troches, capsules, elixirs, suspensions, syrups, wafers, and the like. Oral dosage forms also include modified release, for example immediate release and timed-release, formulations. Examples of modified-release formulations include, for example, sustained-release (SR), extended-release (ER, XR, or XL), time-release or timed-release, controlled-release (CR), or continuous-release (CR or Contin), employed, for example, in the form of a coated tablet, an osmotic delivery device, a coated capsule, a microencapsulated microsphere, an agglomerated particle, e.g., as of molecular sieving type particles, or, a fine hollow permeable fiber bundle, or chopped hollow permeable fibers, agglomerated or held in a fibrous packet. Timed-release compositions can be formulated, e.g. liposomes or those wherein the active compound is protected with differentially degradable coatings, such as by microencapsulation, multiple coatings, etc. Liposome delivery systems, include, for example, small unilamellar vesicles, large unilamellar vesicles and multi lamellar esicles. Liposomes can be formed from a variety of phospholipids, such as cholesterol, stearylamine or phosphatidylcholines.

It is also possible to freeze-dry the compounds of the application and use the lyophilizates obtained, for example, for the preparation of products for injection.

A compound of the application, may also be administered parenterally. Solutions of a compound of the application can be prepared in water suitably mixed with a surfactant such as hydroxypropylcellulose. Dispersions can also be prepared in glycerol, liquid polyethylene glycols, DMSO and mixtures thereof with or without alcohol, and in oils. Under ordinary conditions of storage and use, these preparations contain a preservative to prevent the growth of microorganisms. A person skilled in the art would know how to prepare suitable formulations.

The pharmaceutical forms suitable for injectable use include sterile aqueous solutions or dispersion and sterile powders for the extemporaneous preparation of sterile injectable solutions or dispersions. In all cases, the form must be sterile and must be fluid to the extent that easy syringability exists. As noted above, solutions for use in injectable formulations can be prepared by dissolving the compounds of the invention in Vitamin E TPGS or polyethoxylated castor oil/ethanol.

Compositions for nasal administration may conveniently be formulated as aerosols, drops, gels and powders. Aerosol formulations typically comprise a solution or fine suspension of the active substance in a physiologically acceptable aqueous or non-aqueous solvent and are usually presented in single or multidose quantities in sterile form in a sealed container, which can take the form of a cartridge or refill for use with an atomising device. Alternatively, the sealed container may be a unitary dispensing device such as a single dose nasal inhaler or an aerosol dispenser fitted with a metering valve which is intended for disposal after use. Where the dosage form comprises an aerosol dispenser, it will contain a propellant which can be a compressed gas such as compressed air or an organic propellant such as fluorochlorohydrocarbon. The aerosol dosage forms air or an organic propellant such as fluorochlorohydrocarbon. The aerosol dosage forms can also take the form of a pump-atomizer.

Compositions suitable for buccal or sublingual administration include tablets, lozenges, and pastilles, wherein the active ingredient is formulated with a carrier such as sugar, acacia, tragacanth, or gelatin and glycerine. Compositions for rectal administration are conveniently in the form of suppositories containing a conventional suppository base such as cocoa butter.

Compounds of the application may be used alone or in combination with other known agents useful for treating cancer. When used in combination with other agents useful in treating cancer, it is an embodiment that the compounds of the application are administered contemporaneously with those agents. As used herein, “contemporaneous administration” of two substances to a subject means providing each of the two substances so that they are both biologically active in the individual at the same time. The exact details of the administration will depend on the pharmacokinetics of the two substances in the presence of each other, and can include administering the two substances within a few hours of each other, or even administering one substance within 24 hours of administration of the other, if the pharmacokinetics are suitable. Design of suitable dosing regimens is routine for one skilled in the art. In particular embodiments, two substances will be administered substantially simultaneously, i.e., within minutes of each other, or in a single composition that contains both substances. It is a further embodiment of the present application that a combination of agents is administered to a subject in a non-contemporaneous fashion.

The dosage of compounds of the application can vary depending on many factors such as the pharmacodynamic properties of the compound, the mode of administration, the age, health and weight of the recipient, the nature and extent of the symptoms, the frequency of the treatment and the type of concurrent treatment, if any, and the clearance rate of the compound in the subject to be treated. One of skill in the art can determine the appropriate dosage based on the above factors. Compounds of the application may be administered initially in a suitable dosage that may be adjusted as required, depending on the clinical response. As a representative example, oral dosages of one or more compounds of the application will range between about 1 mg per day to about 1000 mg per day for an adult, suitably about 1 mg per day to about 500 mg per day. In an embodiment of the application, compositions formulated for oral administration and the compounds are suitably in the form of tablets containing 0.25, 0.5, 0.75, 1.0, 5.0, 10.0, 20.0, 25.0, 30.0, 40.0, 50.0, 60.0, 70.0 75.0, 80.0, 90.0, 100.0 150, 200, 250, 300, 350, 400, 450, 500, 550, 600, 650, 700, 750, 800, 850, 900, 950 or 1000 mg of active ingredient per tablet. Compounds of the application may be administered in a single daily dose or the total daily dose may be divided into two, three of four daily doses.

The pharmaceutical compositions may comprise one or more pharmaceutical accepted carriers including but not limited to the following, along with one or more of the compounds of the application:

(a) d-alpha-tocopheryl polyethylene glycol 1000 succinate (Vitamin E TPGS);

### (b) Antibody-Drug Conjugate (ADC);

### (c) Human Serum Albumin (HSA);

### (d) α-Fetoprotein (AFP);

### (e) Polyethylene Glycols (PEGs); and

(f) polyethoxylated castor oil/Ethanol (2:1 v/v to 1:2 v/v, or 1:1 v/v).

### IV. Methods and Uses of the Application

The compounds of the application have been shown to have anti-tumour effects. Surprising, the compounds of the application, have shown improved inhibition of tumour cell growth in several different tumour cell lines compared with the unconjugated alkaloid compounds. This is surprising considering the size of the group being added to the alkaloid compound. The addition of such a long chain would be expected to dramatically alter the properties of the molecule and its ability to orient in space.

Therefore, the compounds of the present application are useful as medicaments. Accordingly the application also includes a compound of the application for use as a medicament.

The present application also includes a method for treating cancer comprising administering a therapeutically effective amount of one or more compounds of the application to a subject in need thereof.

The present application also includes one or more compounds of the application for use to treat cancer. Also included is a use of one or more compounds of the application to treat cancer and a use or one or more of the compounds of the application for preparing a medicament for treating cancer.

In an embodiment of the application, the cancer is selected from pancreatic cancer, lung cancer, colorectal cancer, prostate cancer, breast cancer and cervical cancer.

The following non-limiting examples are illustrative of the present application:

### EXAMPLES

### Example 1

### Isolation of Narciclasine

Natural narciclasine was extracted from various daffodil bulbs as reported in the literature (Dumont P et al., Neoplasia. 2007 9:766; Piozzi F et al., Tetrahedron. 1968 24:1119). The extracts were purified using silica gel chromatography using CH₂Cl₂:CH₃OH (8:2) as eluent. Needle-shaped crystals of narciclasine were obtained by crystallization using methanol:water (1:2). Narciclasine was characterized using ¹H and ¹³C NMR, assayed using HPLC and used as such for synthesizing the conjugates.

### Example 2

### Narciclasine 2-Hydroxy DHA Conjugate—Compound I(a)

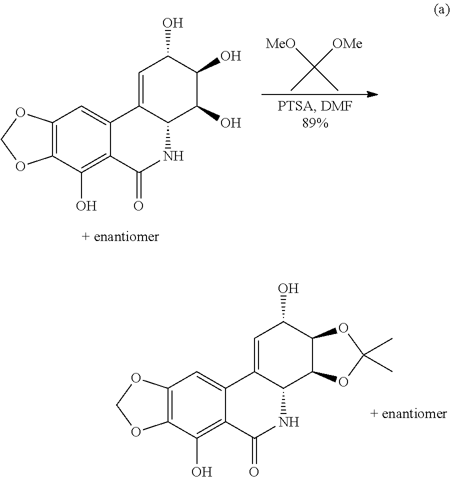

A solution of natural narciclasine (103 mg, 0.336 mmol), 2,2 dimethoxypropane (0.5 mL, 4.08 mmol), and p-toluenesulfonic acid (18 mg, 0.105 mmol) in dimethyl formamide (0.5 mL) was stirred at room temperature for 16 h. Pyridine (0.18 mL, 2.2 mmol) was added to the suspension and the mixture was stirred for one hour at room temperature, after diluting with water (3 mL). The precipitated product was washed with water and dried at 64° C. under high vacuum to give narciclasine 3,4-acetonide (93 mg, yield: 80%) as an amorphous powder.

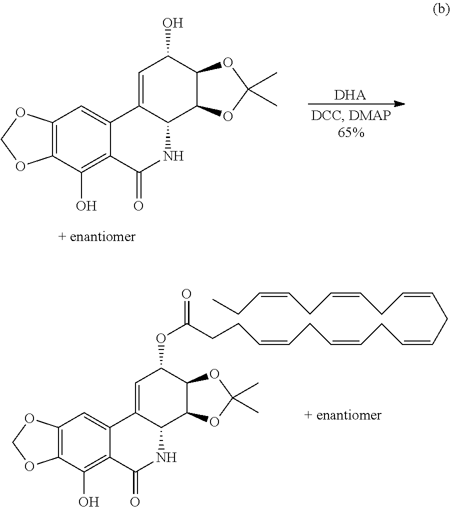 [SMILES: CC1(C)O[C@@H]2[C@H](O1)[C@@H](O)C=C1c3cc4c(c(O)c3C(=O)N[C@H]12)OCO4 ; "DHA" ; CC/C=C\C/C=C\C/C=C\C/C=C\C/C=C\C/C=C\CCC(=O)O[C@H]1C=C2c3cc4c(c(O)c3C(=O)N[C@H]2[C@@H]2OC(C)(C)O[C@@H]21)OCO4]

DHA was purchased from Nu Chek Prep Inc., USA. DHA can be unstable in the presence of oxygen therefore it is stored under nitrogen in sealed vials and all reactions are performed under nitrogen. To a solution of narciclasine-3,4-acetonide (93 mg, 0.27 mmol) in methylene chloride (5 ml) under nitrogen were added 4-dimethylaminopyridine (19 mg, 0.16 mmol), 1,3-dicyclohexylcarbodiimide (61 mg: 0.30 mmol), and DHA (0.11 mL; 0.30 mmol). The reaction mixture was stirred at room temperature for 22 hours. After dilution with methylene chloride (20 mL), the reaction mixture was washed with 5% aqueous hydrochloric acid (10 mL), water (6 mL), and brine (10 mL). The mixture was dried over sodium sulfate, concentrated and purified by column chromatography (silica gel:hexanes/ethyl acetate: 7:3) to give (94 mg, 0.14 mmol, yield: 41%).

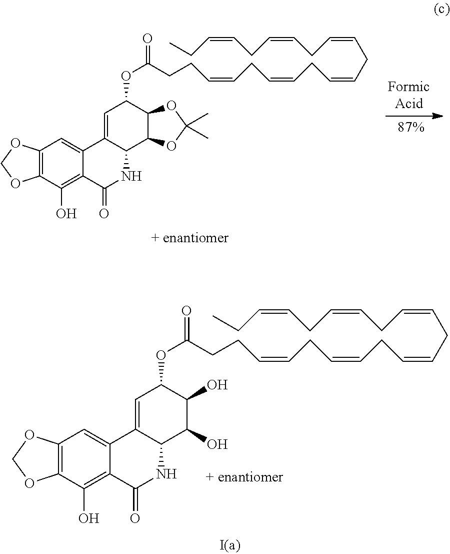 [SMILES: CC/C=C\C/C=C\C/C=C\C/C=C\C/C=C\C/C=C\CCC(=O)O[C@H]1C=C2c3cc4c(c(O)c3C(=O)N[C@H]2[C@@H]2OC(C)(C)O[C@@H]21)OCO4 ; CC/C=C\C/C=C\C/C=C\C/C=C\C/C=C\C/C=C\CCC(=O)O[C@H]1C=C2c3cc4c(c(O)c3C(=O)N[C@H]2[C@H](O)[C@@H]1O)OCO4]

To a solution of narciclasine 3,4-acetonide (94 mg, 0.14 mmol) in methylene chloride (4.0 mL) at 0° C., a cold mixture of formic acid (4.0 mL) and water (80 μL) was added. The reaction mixture was flushed with nitrogen and was stirred at 3 C. for 8 hours and stored in a freezer (−20° C.) overnight. The solution was then stirred at 3° C. for additional 5 hours. The reaction mixture was diluted with ethyl acetate (30 mL) and was washed twice with water (15 mL) and once with brine (15 mL). The solution was dried over anhydrous Na₂SO₄ and evaporated to dryness (keeping the temperature under 25° C.). The residue was dried further under vacuum at room temperature. It was purified with column chromatography (silica gel, methanol/methylene chloride: 5:95) to give white solid of the titled compound (70 mg, 0.11 mmol, yield: 79%).

### Example 3

### Narciclasine 7-Hydroxy DHA Conjugate with Propyl Ether Linker—Compound I(b)

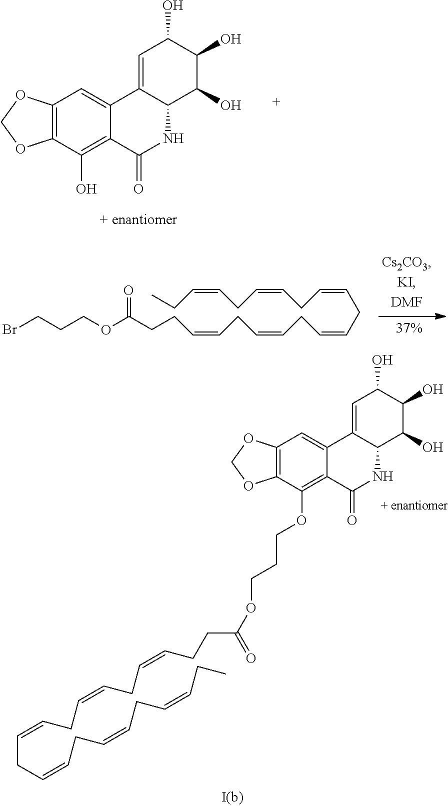

To a solution of natural narciclasine (32.8 mg, 0.107 mmol) in dimethyl formamide (DMF, 1.5 mL) in a test tube, the DHA alkyl bromide (48.04 mg, 1.107 mmol) was added followed by caesium carbonate (34.78 mg, 0.107 mmol) and potassium iodide (3.55 mg, 0.021 mmol). The reaction mixture was flushed with nitrogen and stirred at 30° C. for 2 days. More cesium carbonate (17.39 mg, 0.053 mmol) and potassium iodide (1.78 mg, 0.010 mmol) were added. The reaction mixture was flushed with nitrogen and continued stirring at 30° C. for 3 days. The reaction mixture was diluted with ethyl acetate (30 mL and was washed with water (10 mL) twice and brine. The solution was dried with Na₂SO₄ and evaporated to dryness (keeping the temperature under 35° C.). The residue was dried further under vacuum at room temperature and it was purified with silica gel chromatography, eluting with methanol:methylene chloride (7:93) to give 27 mg of the title compound as a white solid (37%). It was washed with a mixture of ethyl acetate and hexanes to give the analytical sample.

### Example 4

### Narciclasine 7-Hydroxy DHA Conjugate—Compound I(c)

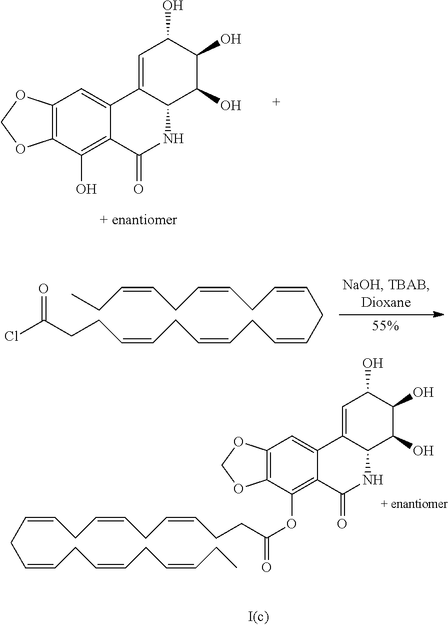 [SMILES: O=C1N[C@@H]2C(=C[C@H](O)[C@@H](O)[C@H]2O)c2cc3c(c(O)c21)OCO3 ; CC/C=C\C/C=C\C/C=C\C/C=C\C/C=C\C/C=C\CCC(=O)Cl ; CC/C=C\C/C=C\C/C=C\C/C=C\C/C=C\C/C=C\CCC(=O)Oc1c2c(cc3c1C(=O)N[C@@H]1C3=C[C@H](O)[C@@H](O)[C@H]1O)OCO2]

DHA (0.080 ml, 0.21 mmol) was dissolved in anhydrous CH₂Cl₂ (0.8 ml) at room temperature. Oxalyl chloride (0.022 ml, 0.25 mmol) was added to the solution and was stirred for two hours. Additional oxalyl chloride (0.022 ml, 0.25 mmol) was added and the solution was stirred for another two hours. The solution was dried under vacuum to yield an oily mass of cis-docosahexaenoic acid chloride (DHA-Cl) that was used as such in next step of the synthesis.

To a solution of narciclasine (50.0 mg, 0.16 mmol) in dioxane (12.5 mL), powdered sodium hydroxide (375 mg, 9.6 mmol) and tetrabutylammonium bisulfate (TBAS, 4.3 mg) was added. A solution of cis-docosahexaenoic acid chloride (DHA-C1) (22.53 mg, 0.065 mmol) in dioxane (2.5 mL) was added dropwise for 15 minutes. The reaction was quenched with 1 mL of acetic acid. The reaction mixture was diluted with 20 mL of ethyl acetate, washed with water (25 mL×6), with brine and dried over anhydrous sodium sulfate. The solution was evaporated to dryness and was further purified using silica gel column, eluting with methanol:methylene chloride (8:92) to give 15 mg of the title compound as a white solid (yield: 15%).

### Example 5

### Preparation of I(a) Composition in Vitamin E TPGS

The long chain fatty acid component of I(a) lowers the drug's solubility in water but increases its solubility to certain pharmaceutical carriers. Vitamin E d-alpha-tocopheryl polyethylene glycol 1000 succinate (Vitamin E TPGS) is both fat and water soluble and was used in making a composition of I(a) for further cell line testing. In this example, Vitamin E TPGS was used in the formation of a pharmaceutical formulation to be used as an injectable. It is of course understood that modifications, alterations and adaptations that may occur are intended to be within the scope of the current application.

Vitamin E TPGS (δ-alpha-tocopheryl polyethylene glycol 1000 succinate) from Isochem, France was used in all the formulations discussed below.

Fifteen to one hundred (15:100) ratio by weight of compound I(a) to Vitamin E TPGS was placed into a glass vial and heated to approximately 45° C. for one hour. A stir bar was then added and the solution was continuously mixed while at 45° C. for a minimum of 2 hours to allow full saturation of the product. Water was then added to the mixture with a ratio of 8 ml per 100 mg of Vitamin E. The solution was left for a minimum of 4 h to allow all vitamin E/I(a) solution to be dissolved. The mixture was then cooled to room temperature, and centrifuged to separate the undissolved material. The centrifugate was assayed for the amount of compound I(a) using HPLC. The resultant formulation was found to contain approximately 1.8-1.9 mg/mL of I(a). This was then diluted to 1.25 mg/ml with a vitamin E solution.

For a 300 mg dose of compound I(a) at a concentration of 1.25 mg/ml and 12.5 mg/ml of vitamin E will give a total of 3 grams of vitamin E.

Vitamin E has been reported to be well tolerated in humans at doses up to 2.3 g m⁻² for 9 consecutive days given intravenously. This results in 4.37 g and 3.68 g for an adult male and female with the average area of 1.9 m² and 1.6 m² respectively.

The administration of compounds to humans or other animals that require the compound for treatment depends on many factors including but not limited to the disease, stage of disease, host recipient, age, weight and frequency of treatment. The preceding describes possible methods of preparation of some of the compounds. This description is intended for illustrative purposes only; the actual method and dosages to be administered or delivered may vary.

### Example 6

### Preparation of I(a) Composition in Cremophor™ EL:Ethanol

As mentioned in Example 5, the fatty acid component of I(a) increases the solubility of the drug in certain organic solvents. Cremophor EL (ethoxylated castor oil, CrEL) is a non-ionic emulsifier that is used to assist in the solubilization of hydrophobic drugs. In this example CrEL from BASF, Germany was used for the formulation discussed below.

One to sixty (1:60) ratio by weight of compound I(a) to CrEL was mixed into a glass vial and stirred for about one hour at room temperature to allow solubilization. Dehydrated ethanol USP was then added at a ratio (v/v) one to one (1:1) to CrEL. The mixture was stirred for half hour to ensure the dissolution of the mixture. The final solution was then tested by HPLC to verify compound I(a) concentration. The resultant formulation was found to contain 9.9 mg/ml of I(a).

### Example 7

### In Vitro Testing on Various Tumour Cell Lines

### (a) Materials and Methods

A549 (CCL-185), BxPC-3 (CRL-1687), HeLa (CCL-2), LoVo (CCL-229), MCF-7 (HTB-22), and PC-3 (CRL-1435) cells were purchased from ATCC (Manassas, Va.). Alamar Blue was purchased from Invitrogen Canada Inc. (Burlington, ON). All other tissue culture and reagent grade materials were purchased from either VWR (Mississauga, ON) or Sigma Aldrich (Oakville, ON).

All cell lines were maintained at 37° C. in a CO₂ cell culture incubator with 5% CO₂ and 95% humidity. A549, Lovo, and PC-3 cells were cultured in F-12K media, BxPC-3 cells were culture in RPMI-1640 media, and HeLa and MCF-7 cells were cultured in Eagle's Minimum Essential Media (MEM) with non-amino acids. All cell culture media was supplemented with 10% fetal bovine serum (FBS), 100 U/L Penicillin, 0.1 mg/mL Streptomycin, 2.5 μg/mL Amphotericin B, and 50 μg/mL Gentamycin. In addition, MEM media for MCF-7 cells also contained 0.01 mg/mL bovine insulin.

All cell lines were plated in black, tissue culture treated, 96 well plates at a cell density of 1×10⁵ celis/mL (1×10⁴ cells/well). Cells were allowed 24 h to attach to the plate prior to being dosed with drug. On the day of the experiment cells were dosed with either a vitamin E vehicle, a DMSO vehicle, I(a) formulated in vitamin E, I(a) formulated in DMSO or narciclsine formulated in DMSO in triplicate. Cells were allowed to incubate for 48 or 72 h. Following incubation cells were stained with Alamar Blue such that the final concentration of Alamar Blue in the test well was 10%. Cells were stained for 2 h and then fluorescence was measured on a Molecular Devices Analyst GT multimode plate reader using an excitation filter of 530/25 nm and an emission filter of 580/10 nm and a 561 nm dichroic mirror. Cell viability was expressed as a percentage of untreated control cells. Data are expressed as mean±standard error and are the average of four independent cell preparations.

The administration of the compounds to humans or other animals that require the drug for treatment depends on many factors including but not limited to the disease, stage of disease, host recipient, age, weight and frequency of treatment. The following describes possible methods of preparation of some of the compounds. This description is intended for illustrative purposes only; the actual method and dosages to be administered or delivered may vary.

### (b) Results

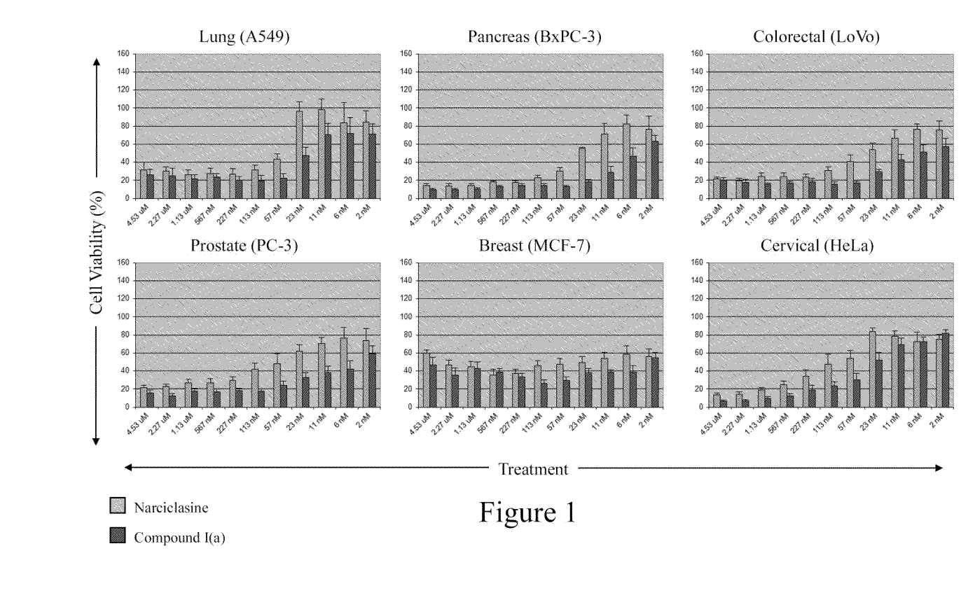

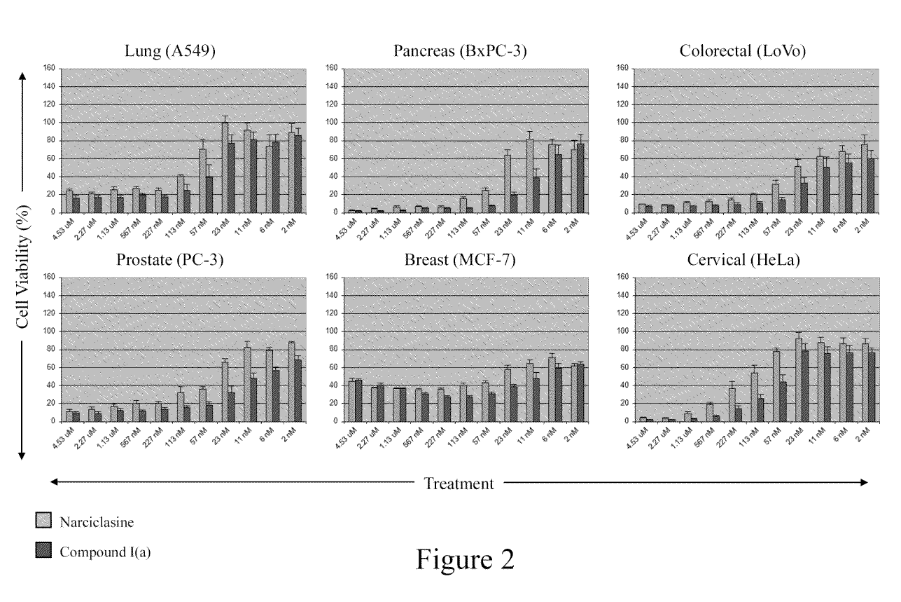

A comparative study of compound I(a) and narciclasine at various concentrations against various tumour cell lines shows that compound I(a) has a similar if not greater effect in vitro against tumour cells compared to its parent compound narciclasine. Cell viability of these tumour cells at various concentrations can be seen on FIG. 1 at 48 hours and FIG. 2 after 72 hours.

After 48 hours it can be seen that compound I(a) as well as narciclasine has a strong anti-cancer effect against a wide variety of cancers. Specifically there is a strong effect on pancreas, colorectal, prostate and cervical tumour cell lines.

A clearer result is visible after 72 hours when compound I(a) and narciclasine had the greatest effect on the tumour cells. Compound I(a) works well against pancreas and colorectal cancer as well as good results on lung, prostate and cervical tumour cells.

### Example 8

### Activity of I(a)/Vitamin E Composition in Tumour Cell Lines

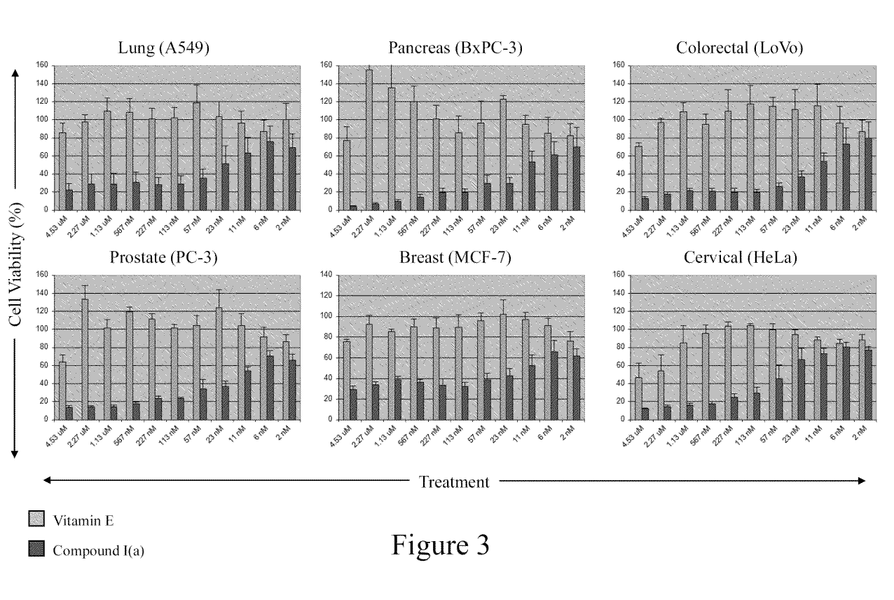

This formulation was tested against the same six cancer cell lines at the same concentrations as compound I(a) using the procedure described in Example 5. The results are shown in FIGS. 3 (48 h) and 4 (72 h) along with comparison values for vitamin E alone. Concentrations of Vitamin E which correspond to each drug treatment are provided in Table 1.

Compound I(a) in a vitamin E solution had a similar effect on tumour cells to compound I(a) in DMSO. The mode of transport had no clear effect on cell viability in vitro. The effect of Vitamin E TPGS in water is negligible at most concentrations. There is a small amount of cell death with Vitamin E TPGS at the highest concentration.

### Example 9

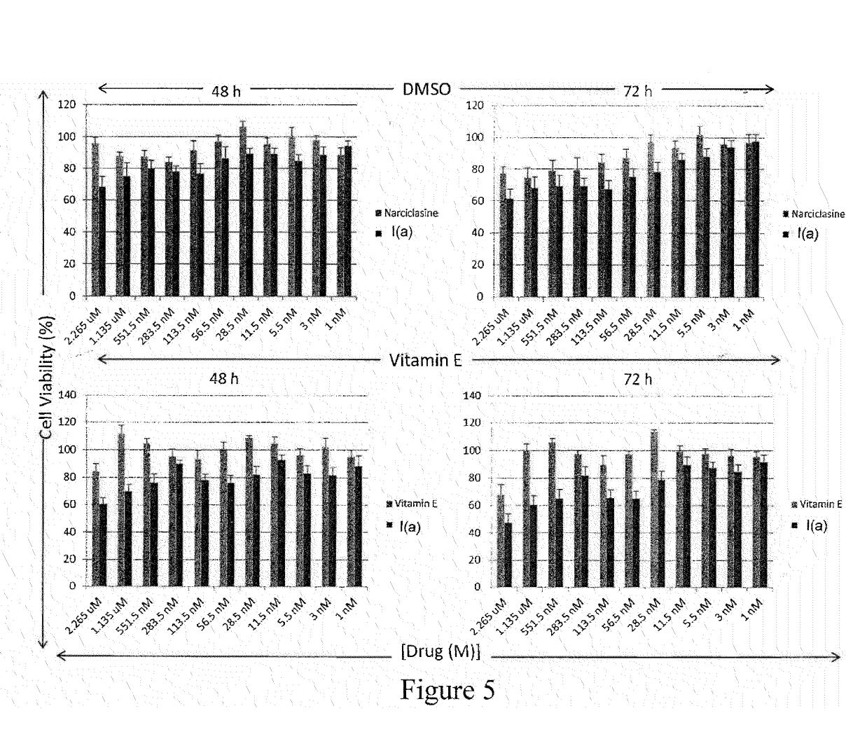

Healthy PBMC (Peripheral Blood Mononuclear Cells) were treated similarly to the above tumour cell line tests. The top two graphs in FIG. 5 consist of compound I(a) and narciclasine in DMSO at various concentrations. At 48 and 72 hours there was a small increase of cell death from compound I(a) compared to narciclasine but the overall cell viability, even at the highest concentration of 2.265 uM, was over 60% and, at the lowest concentration, around 90%. The second PBMC test contained compound I(a) in a vitamin E formulation compared to a vitamin E solution (bottom 2 graphs, FIG. 5). Vitamin E had a minimal effect on healthy cells with a range of viability from 85% to 110%. There was a similar effect with compound I(a) in vitamin E compared to compound I(a) in DMSO. Comparing PBMC cell line tests to the tumour cell line tests where cell viability ranged from 5% to 80% from highest to lowest concentrations it can be safely concluded that compound I(a) and narciclasine selectively kill cancer cells at a much higher percentage than healthy cells.

While the present disclosure has been described with reference to what are presently considered to be the preferred examples, it is to be understood that the disclosure is not limited to the disclosed examples. To the contrary, the disclosure is intended to cover various modifications and equivalent arrangements included within the spirit and scope of the appended claims.

TABLE 1

| Drug | VIT E TPGS | Vitamin E |
|---|---|---|
| Concentration | (mg/ml) | TPGS (%) |
| 4.53 uM | 0.028 | 0.0028 |
| 2.27 uM | 0.014 | 0.0014 |
| 1.13 uM | 0.007 | 0.0007 |
| 567 nM | 0.0035 | 0.00035 |
| 227 nM | 0.0014 | 0.00014 |
| 113 nM | 0.0007 | 0.00007 |
| 57 nM | 0.00035 | 0.000035 |
| 23 nM | 0.00014 | 0.000014 |
| 11 nM | 0.00007 | 0.000007 |
| 6 nM | 0.000035 | 0.0000035 |
| 2 nM | 0.000014 | 0.0000014 |
| 1 nM | 0.000007 | 0.0000007 |

1. A compound of Formula I: 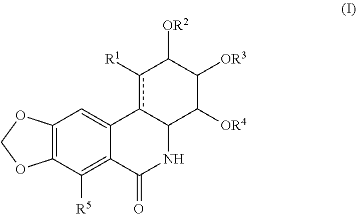
wherein
R¹ and R⁵, are independently, H, OH or O-(L)ₙ-B;
R², R³ and R⁴, are independently, H or -(L)ₙ-B;
provided that one, two or three of R¹, R², R³, R⁴ and R⁵ comprise -(L)ₙ-B;
L is a linker group;
B is a lipophilic biomolecule;
n is 0 or 1; and [symbol]
is a single or a double bond,
or a stereoisomer thereof, or a pharmaceutically acceptable salt and/or solvate.

2. (canceled)

3. The compound of claim 1, wherein, R¹ is H, R² is O-(L)ₙ-B, R³ is OH, R⁴ is OH, R⁵ is OH and [symbol] is a double bond to provide narciclasine 2-position conjugate.

4. The compound of claim 1, wherein R¹ is H, R² is OH, R³ is OH, R⁴ is OH, R⁵ is O-(L)ₙ-B and [symbol] is a double bond to provide a narciclasine 7-position conjugate.

5. The compound of claim 1, wherein R¹ is H, R² is O-(L)ₙ-B, R³ is OH, R⁴ is OH, R⁵ is and [symbol] is a double bond to provide Iycoridine 2-position conjugate.

6. The compound of claim 1, wherein R¹ is OH, R² is O-(L)ₙ-B, R³ is OH, R⁴ is OH, R⁵ is OH and [symbol] is a single bond to provide pancratistatin 2-position conjugate.

7. The compound of claim 1, wherein R¹ is OH, R² is OH, R³ is OH, R⁴ is OH, R⁵ is O-(L)ₙ-B and [symbol] is a single bond to provide a pancratistatin 7-position conjugate.

8. The compound of claim 1, wherein the compound of Formula I has the following relative stereochemistry when [symbol] is a double bond: 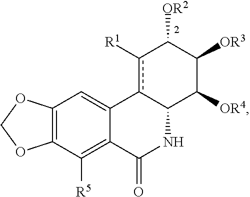
and the following relative stereochemistry when [symbol] is a single bond:

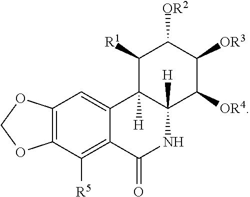

9. (canceled)

10. The compound of claim 1, wherein L is selected from C₁₋₁₀alkylene in which one or more of the CH₂ groups is optionally replaced with C(O)O or O.

11. (canceled)

12. The compound of claim 1, wherein n is 0.

13. (canceled)

14. The compound of claim 1, wherein B is a long chain fatty acid.

15. The compound of claim 14, wherein the fatty acid is a C₁₂₋₂₂ fatty acid.

16. The compound of claim 14, wherein B is selected from cis-docosahexaenoic acid (DHA), oleic acid, lauric acid, palmitic acid, Iinoelaidic acid, vaccenic acid, palmitoleic, myristoleic, a-linolenic acid (ALA), arachidonic acid, eicosapentaenoic acid (EPA) and eruric acid.

17. The compound of claim 16, wherein B is selected from cis-docosahexaenoic acid (DHA), EPA, ALA, lauric acid and oleic acid.

18. The compound of claim 17, wherein B is DHA.

19. (canceled)

20. (canceled)

21. (canceled)

22. The compound of claim 1, wherein B is a monoclonal antibody.

23. A pharmaceutical composition comprising one or more compounds of claim 1 and a pharmaceutically acceptable carrier.

24. The composition of claim 23, wherein the compounds are formulated into a composition comprising vitamin E δ-alpha-tocopheryl polyethylene glycol 1000 succinate (Vitamin E TPGS), as a carrier.

25. The composition of claim 24, wherein the ratio of the one or more compounds to Vitamin E TPGS is about 1:50 to about 1:10, about 1:20 to about 1:15 or about 3:20.

26. (canceled)

27. A method for treating cancer comprising administering a therapeutically effective amount of one or more compounds of claim 1 to a subject in need thereof.

28. The method of claim 27, wherein the cancer is selected from pancreatic cancer, lung cancer, colorectal cancer, prostate cancer, breast cancer and cervical cancer.
